(19) United States
(12) Patent Application Publication
(10) Pub. No.: US 2026/0066043 A1
(43) Pub. Date: Mar. 5, 2026

(54) SYSTEMS AND METHODS FOR RANKING CELLS

(71) Applicant: L'OREAL, Paris (FR)

(72) Inventors: Patrick Morley WILLOUGHBY, New York, NY (US); I-Chien Liao, Princeton, NJ (US); Rebecca Barresi, Robbinsville, NJ (US); Ying Chen, Montville, NJ (US)

(21) Appl. No.: 18/820,039

(22) Filed: Aug. 29, 2024

(51) Int. Cl. G16B 25/00; G16H 20/10

(52) U.S. Cl. CPC G16B 25/00; G16H 20/10

(57) ABSTRACT

Systems, apparatuses, methods, and computer program products are disclosed for ranking cells in reference to a cell or biological signature associated with skin, hair or scalp health. In an embodiment, the method may include identifying cells of interest of a subject. The method may or may not include iteratively applying an external stimuli to induce a pathological state different portions of cells. The method may include analyzing, via an OMICS sequencer, each portion of the cells to produce a multi-marker OMICS analysis. The method may include determining a rank for each of the cells for one or more selected characteristics based on application of the multi-marker OMICS analysis to an expression model for the one or more selected characteristics. The method may include determining a next action based on a the rank for each of the cells.

### TECHNOLOGICAL FIELD

Example embodiments of the present disclosure relate generally to systems and methods for ranking cells and, particularly to systems and methods for ranking cells in reference to a cell or biological signature or characteristic associated with skin, hair, or scalp health, among other cells from other areas.

### BACKGROUND

Currently, ribonucleic acid (RNA) analysis or sequencing, as well as other OMICS analysis or sequencing, sequencing produces varying types of charts or data. These charts and data may include heatmaps and/or other types of charts representing different types of gene expressions, such as Z-score gene expression or a differential gene expression. Further, the gene set may be a senescence curated gene set, which may be biased.

Additionally, the output of OMICS analysis is, typically, not easily interpretable given transcriptomic heterogeneity and variability. In other words, interpreting the resulting output from OMICS analysis may require a user with a higher than typical level of expertise. Further, such an interpretation, even with an expert user, still takes significant time and effort, as well as potentially including bias. Thus, even if a user correctly interprets the resulting analysis, user bias may cause incorrect interpretations.

### BRIEF SUMMARY

As noted, typical ribonucleic acid (RNA) analysis and/or -OMICS sequencing results in an output that is difficult to interpret and may include bias. Thus, there is felt a need to limit and solve the aforementioned problems and drawbacks and provide systems and methods for scoring and/or ranking cells exhibiting diverse phenotypes, particularly cells exhibiting enrichment or depletion of sets of genes associated with varying pathological states, either endogenous or induced, and for determining a correlation between each analyzed cell and the one or more selected characteristics based on a cell signature, thus increasing interpretability and enabling faster and meaningful adjustments to product formula and/or enabling recommended use of a product to increase/decrease a characteristic caused by the product (for example, decrease in senescence or biological aging). Such systems and methods, as well as apparatus and computer programs or products, may obtain data from-OMICS sequencing of cells (for example using genomics sequencing, transcriptomics sequencing, proteomics sequencing, epigenomics sequencing, lipidomics sequencing, or metabolomics sequencing or other types of OMICS sequencing or other tests)exhibiting one or more characteristics (for example, one or more of cells subjected to an external stimuli or cells exhibiting one or more of a plurality of selected cell characteristics). As noted, the selected cells may have been subjected to a selected stimuli over a selected period of time. Further, the cells may be obtained from varying areas of a subject, for example, a first set of cells may be collected from skin around a subject's eyes, while a second set of cells may be collected from skin around the subject's mouth, among other areas. Further still, the cell characteristics may include, for example, a subject's age, race, gender, other demographic data, among other characteristics. The OMICS sequencing may be based on one or more characteristics of the cells, one or more gene characteristics, senescence scores, single cell transcriptomic measurements using RNA Multiplexed error-robust fluorescence in situ hybridization (MERFISH), and/or on another cell characteristic test or other OMICS test, as will be understood by one skilled in the art. Once the data has been obtained, such systems and methods may score and/or rank each cell or cells for one or more selected characteristics. Based on the scores and/or rank, the systems and methods may suggest an updated product formula and/or recommend decreased or increased use of a product to increase/decrease skin, scalp, and hair health/pathological state characteristic caused by the product (for example, decrease in senescence or biological aging).

Accordingly, an embodiment of the disclosure is directed to a method for ranking a cells of interest. The method may include identifying cells of interest of a subject. The method may include analyzing, via a ribonucleic acid (RNA) sequencer and other-OMICS sequencing technologies, each portion of the cells to produce a multi-marker OMICS analysis. The method may include determining a rank for each of the cells for one or more selected characteristics based on application of the multi-marker-OMICS analysis to an expression model for the one or more selected characteristics. The method may include determining cell enrichment for each analyzed cells based on the rank for each of the cells. In another embodiment, the method may include determining a next action based on the rank for each of the cells.

In an embodiment, determining the rank comprises converting the multi-marker genetic or -OMICS analysis to an array of values, removing identifiers from the array of values, determining an average or median of the array of values and a number of instances in the array of values, applying the array of values to assigned pair ranked test to generate a score for each value in the array of values based on a signature of the one or more selected characteristics, and re-assigning the identifiers to the array of values.

In an embodiment, the OMICS sequencer may include one of a genomics sequencer, transcriptomics sequencer, proteomics sequencer, epigenomics sequencer, lipidomics sequencer, or metabolomics sequencer. The multi-marker OMICS analysis may include one of transcriptomics data, proteomics data, epigenomics data, genomics data, lipidomics data, or metabolomics data. The cells of interest may include one or more of skin cells, skin tissue, hair cells, scalp cells, scalp tissue, reconstructed tissue, or ex vivo skin. The one or more cells of interest may be collected from one or more subjects of one or more different ages, one or more difference races, one or more genders.

In an embodiment the method may include, in response to determination of the rank for each of the plurality of cells (or, in some examples, samples), generating a user interface. The method may include displaying each of the cells based on ranking. The method may also include displaying data indicative of the correlation between each analyzed cell and the one or more selected characteristics based on a cell signature.

In an embodiment, the next action may comprise one or more of determining a correlation between each analyzed cell and the one or more selected characteristic, suggesting use of a selected product or agent, designing a personalized mixture or product, or prescribing a selected product.

In yet another embodiment, the next action may include one or more of determining a correlation between each analyzed cell and the one or more selected characteristic, suggesting use of a selected product or agent, designing a personalized mixture or product, or prescribing a selected product

In an embodiment, the one or more selected characteristics may include one or more of differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health.

In another embodiment, prior to analysis of each portion of the cells, the method may include iteratively applying different stimuli to different portion of the cells. The stimuli may induce or may cause a pathological state for each different portion of cells. The stimuli may include one or more of exposure to sunlight, exposure to ultraviolet light, exposure to environmental pollutants, exposure to chemicals, exposure to antibodies, exposure to vesicles, exposure to genetic stimuli, exposure to a selected temperature, exposure to a selected pressure, or a selected time.

Another embodiment of the disclosure is directed to an apparatus for ranking a cells of interest. The apparatus may include an analysis circuitry. The analysis circuitry may be configured to obtain a multi-marker OMICS analysis of a plurality of cells. each of the plurality of cells may exhibit one or more characteristics. The apparatus may include a ranking circuitry. The ranking circuitry may be configured to determine a rank for each of the cells for one or more selected characteristics based on application of the multi-marker OMICS analysis to an expression model for the one or more selected characteristics. The ranking circuitry may be configured to determine a cell enrichment for each analyzed cells based on the rank for each of the cells.

In another embodiment, the apparatus may further include an OMICS sequencer. The OMICS sequencer may be configured to analyze cells to produce the multi-marker OMICS analysis. The analysis circuitry may communicatively connect to the OMICS sequencer and the analysis circuitry may be configured to, in response to reception of cells of interest at the OMICS sequencer, prompt the OMICS sequencer to analyze the cells of interest.

In another embodiment, the apparatus may include a user interface communicatively connected to the analysis circuitry. The analysis circuitry may obtain the multi-marker OMICS analysis from the user interface.

In an embodiment, the cells of interest may comprise cells obtained from a subject. Additionally, the ranking circuitry may be further configured to the ranking circuitry is further configured to determine a recommended use suggestion of a selected product based on history of use by the subject.

Another embodiment of the disclosure is directed to a computer program product for ranking a cells of interest. The computer program product may include a non-transitory machine-readable storage medium storing software instructions that, when executed, cause an apparatus to obtain a multi-marker OMICS analysis of a plurality of cells. Each of the plurality of cells may include one or more of cells subjected to an external stimuli or cells exhibiting one or more of a plurality of selected cell characteristics. The non-transitory machine-readable storage medium may store additional software instructions that, when executed, determine a rank for each of the cells for one or more selected characteristics based on application of the multi-marker OMICS analysis to an expression model for the one or more selected characteristics.

In an embodiment, the non-transitory machine-readable storage medium may store additional software instructions that, when executed, determine a recommended use suggestion of a selected product based on history of use on the cells. In another embodiment, the multi-marker OMICS analysis may comprise one or more of a heatmap, a cell or sample by OMICS matrix, or a cell type distribution.

The foregoing brief summary is provided merely for purposes of summarizing example embodiments illustrating some aspects of the present disclosure. Accordingly, it will be appreciated that the above-described embodiments are merely examples and should not be construed to narrow the scope of the present disclosure in any way. It will be appreciated that the scope of the present disclosure encompasses many potential embodiments in addition to those summarized above, some of which will be described in further detail below.

### BRIEF DESCRIPTION OF THE FIGURES

Having described certain example embodiments of the present disclosure in general terms above, reference will now be made to the accompanying drawings, which are not necessarily drawn to scale. Some embodiments may include fewer or more components than those shown in the figures.

FIG. 1A and FIG. 1B illustrate systems in which some example embodiments may be used for scoring and/or ranking each of a plurality of cells.

FIG. 2 illustrates a schematic block diagram of example circuitry embodying a device that may perform various operations in accordance with some example embodiments described herein.

FIG. 3A and FIG. 3B illustrate example user interfaces used in some example embodiments described herein.

FIG. 4 illustrates an example flowchart for scoring and/or ranking each of a plurality of cells, in accordance with some example embodiments described herein.

### DETAILED DESCRIPTION

Some embodiments of the present disclosure will now be described more fully hereinafter with reference to the accompanying figures, in which some, but not all, embodiments of the disclosures are shown. Indeed, these disclosures may be embodied in many different forms and should not be construed as limited to the embodiments set forth herein; rather, these embodiments are provided so that this disclosure will satisfy applicable legal requirements.

The term “computing device” is used herein to refer to any one or all of programmable logic controllers (PLCs), programmable automation controllers (PACs), industrial computers, desktop computers, personal data assistants (PDAs), laptop computers, tablet computers, smart books, palm-top computers, personal computers, smartphones, wearable devices (such as headsets, smartwatches, or the like), and similar electronic devices equipped with at least a processor and any other physical components necessarily to perform the various operations described herein. Devices such as smartphones, laptop computers, tablet computers, and wearable devices are generally collectively referred to as mobile devices.

The term “server” or “server device” is used to refer to any computing device capable of functioning as a server, such as a master exchange server, web server, mail server, document server, or any other type of server. A server may be a dedicated computing device or a server module (e.g., an application) hosted by a computing device that causes the computing device to operate as a server. A server module (e.g., server application) may be a full function server module, or a light or secondary server module (e.g., light or secondary server application) that is configured to provide synchronization services among the dynamic databases on computing devices. A light server or secondary server may be a slimmed-down version of server type functionality that can be implemented on a computing device, such as a smart phone, thereby enabling it to function as an Internet server (e.g., an enterprise e-mail server) only to the extent necessary to provide the functionality described herein.

As used herein, a “non-transitory machine-readable storage medium” or “memory” may be any electronic, magnetic, optical, or other physical storage apparatus to contain or store information such as executable instructions, data, and the like. For example, any machine-readable storage medium described herein may be any of random access memory (RAM), volatile memory, non-volatile memory, flash memory, a storage drive (e.g., hard drive), a solid state drive, any type of storage disc, and the like, or a combination thereof. The memory may store or include instructions executable by the processor.

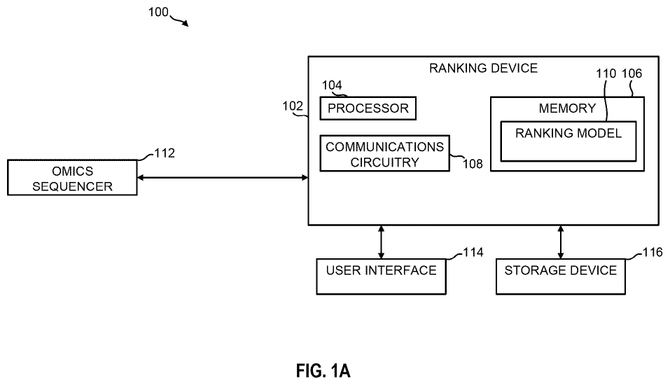

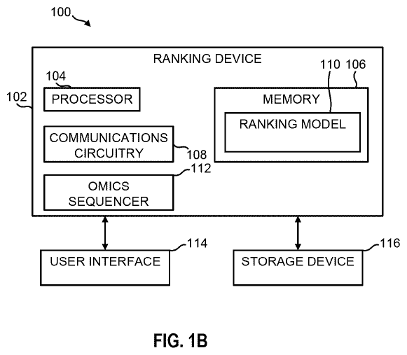

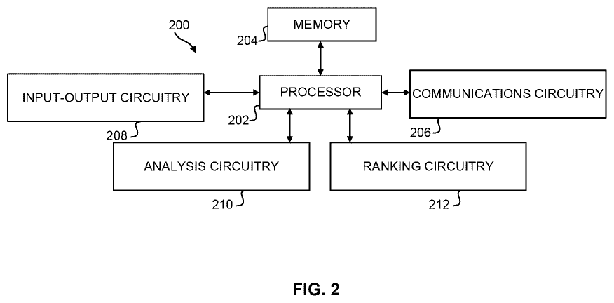

As used herein, a “processor” or “processing circuitry” may include, for example one processor or multiple processors included in a single device or distributed across multiple computing devices. The processor (such as, processor 104 and processor 202 shown in FIGS. 1A through 2) may be at least one of a central processing unit (CPU), a semiconductor-based microprocessor, a graphics processing unit (GPU), a field-programmable gate array (FPGA) to retrieve and execute instructions, a real time processor (RTP), other electronic circuitry suitable for the retrieval and execution instructions stored on a machine-readable storage medium, or a combination thereof.

As noted above, methods, apparatuses, systems, and computer program products are described herein that provide scores and/or ranking for analysis of a plurality of cells. Each of the cells or different sets of cells or singular cells or samples may exhibit one or more selected characteristics. For example, some or a selected amount of the cells may be exposed to external stimuli to induce one or more pathological health states, while some of the cells or singular cells or samples may not be exposed. The external stimuli may include sunlight (for example, cells exposed to sunlight), ultraviolet light (for example, cells exposed to ultraviolet light), a selected temperature (for example, cells exposed to the selected temperatures), exposure to a selected pressure (for example, cells exposed to the selected pressure), or a selected time (for example, a time that the selected cells are exposed to some other stimuli or an amount of time after a pathological health state is induced). In another example, the cells may exhibit other characteristics. For example, the cells may be obtained from a plurality of subjects of a different ages, races, genders, family history, and/or other demographic characteristic or quality. Further, cells may be collected from subjects exhibiting one of a selected medical condition, a disease, or healthy conditions. Further, the cells may be obtained from a plurality of locations of the subject, for example, selected portions or areas of the face (such as, around the eyes or mouth), areas of the scalp, and/or other areas of the subject's body.

Further, the analysis and ranking may correspond to the one or more selected characteristics, such as senescence, aging, skin quality, scalp health, hair health, hair quality, and/or other characteristics related to skin, scalp and hair. Further, such systems may recommend decreased or increased usage of a particular product and/or suggest adjustments to a product formula. Further, the systems may determine a customized or personalized formula and/or product, may determine a treatment regimen to increase or decrease one of the selected characteristics, and/or may prescribe a selected product. Further, such systems may correlate cells to a plurality of characteristics. For example, such systems may determine that cells of a particular age exhibit higher levels of senescence, as opposed to cells of a different age. Traditionally, the output of RNA sequencers or -OMICS sequencers or analyzers may be difficult to interpret and may require user expertise or, at least, an experienced user. In addition, typically, such analysis and/or interpretation includes some level of bias, potentially skewing the end interpretation. Thus, ranking cells according to some characteristic by a user could produce skewed or biased results, or, in other embodiments, results that include human-based errors.

In contrast to these conventional techniques for interpreting sequencing data, the present disclosure describes scoring and/or ranking a plurality of cells (for example, cells of interest). Further, based on such scoring and/or ranking, the systems and methods described herein may determine a next action, as described herein. Such systems and methods may include or may connect to an OMICS sequencer or analyzer.

As the OMICS sequencer or analyzer analyzes a plurality of samples (in other words, a plurality of cells exhibiting one or more characteristics), the resulting data may be transmitted to a computing device, scoring or ranking device or system, and/or a scoring or ranking apparatus. The OMICS sequencer may perform such an analysis in relation to or based on one or more characteristics of the cells, one or more gene characteristics, senescence scores, single cell transcriptomic measurements using RNA Multiplexed error-robust fluorescence in situ hybridization (MERFISH), and/or on another cell characteristic or other OMICS test, as will be understood by one skilled in the art. The OMICS analyzer may include one or more of a genomics sequencer, transcriptomics sequencer, proteomics sequencer, epigenomics sequencer, lipidomics sequencer, or metabolomics sequencer. The OMICS analyzer may produce a multi-marker OMICS analysis. The multi-marker OMICS analysis may include one or more of transcriptomics data, proteomics data, epigenomics data, genomics data, lipidomics data, or metabolomics data. The cells of interest may include one or more of skin cells, skin tissue, hair cells, scalp cells, scalp tissue, reconstructed tissue, or ex vivo skin. In other embodiments, the computing device, scoring or ranking device or system, and/or a scoring or ranking apparatus may obtain data from or via a user interface and/or from a database or other storage or memory. The computing device, scoring device or system, and/or a scoring apparatus may then score each of the analyzed cells. Based on those scores, the computing device, scoring or ranking device or system, and/or a scoring or ranking apparatus may determine an adjustment to a product and/or recommend a decrease or increase in use of a selected product. As noted, the computing device, scoring or ranking device or system, and/or a scoring or ranking apparatus may, in addition to scores, rank the cells.

Accordingly, the present disclosure sets forth systems, methods, and apparatuses that provide easily interpretable analysis, as well as recommendations or next actions based on that analysis, based on data from an OMICS sequencer, which is typically difficult to interpret and may require users with significant expertise. There are many advantages of these and other embodiments described herein. For instance, the resulting scores and/or ranking enable a user to easily determine whether a cell or series of cells exhibit overlap or correspond to a cell or gene expression signature, the cell or gene expression signature corresponding to some selected characteristics (for example, anti-aging). For example, a user may quickly and easily determine whether a series of cells corresponding to different areas of one or more users of a selected age experience senescence at selected rates. In addition, the systems and methods described herein may provide suggested or personalized formula, products and/or, regimens based on the ranking and/or scoring.

Although a high level explanation of the operations of example embodiments has been provided above, specific details regarding the configuration of such example embodiments are provided below.

Example embodiments described herein may be implemented using any of a variety of computing devices or servers. To this end, FIG. 1A and FIG. 1B illustrate an example environment within which embodiments of the present disclosure may operate. As illustrated, a ranking system 100 may include a ranking device 102. The ranking device 102 may include a processor 104, a memory 106, and/or a communications circuitry 108. The memory 106 may include instructions and/or other software or algorithms, such as a ranking model 110. The ranking device 102 may connect, via the communication circuitry 108 and/or a communications network, to an OMICS sequencer 112, a user interface 114, and/or a storage device 116, among other devices and/or components. As illustrated and/or in other implementations, the ranking system 100, and any constituent device(s) and/or storage device(s) 116 may receive and/or transmit information via a communications network (for example, the internet and/or an intranet) with any number of other devices.

The ranking device 102 may be implemented as one or more servers, which may or may not be physically proximate to other components of the ranking system 100. Furthermore, some components of ranking device 102 may be physically proximate to the other components of the ranking system 100 while other components are not. Ranking device 102 may receive, process, generate, and transmit data, signals, and electronic information to facilitate the operations of the ranking system 100. Particular components of ranking device 102 are described in greater detail below with reference to apparatus 200 in connection with FIG. 2.

As noted, the memory 106 may include instructions and/or other algorithms. Such instructions, when executed by the processor 104, may control various functions and/or aspects of the ranking device 102. For example, a user may, via the user interface 114, initiate a scoring and/or ranking operation. Upon initiation and/or upon entry or input of one or more samples into the OMICS sequencer 112, the ranking device 102 may cause the OMICS sequencer 112 to analyze a plurality of cell samples. Each cell sample may exhibit one or more characteristics. For example, the cells may be exposed to stimuli to induce a pathological state, such stimuli including, for example, ultra-violet light, chemicals, environmental pollutants, antibodies, vesicles, genetic stimuli, a selected temperature, pressure, time, and/or some other stimuli=and/or may be unexposed. Such exposure may have occurred for some selected period of time prior to collection of the cells, such as for a series of days prior to collection of the sample or some time more or less than a series of days. The OMICS sequencer 112 may produce data corresponding to one or more characteristics, gene expressions (for example, enrichment, depletion, and/or other types of gene expressions), and/or other factors of the plurality of cells. For example, the OMICS sequencer 112 may produce a cell-by-gene matrix, gene expressions, and/or other data in various formats. For example, the OMICS sequencer 112 may generate skin barrier cell values upon analysis. In another embodiment, the ranking device 102 may receive or obtain data related to cell analysis from the storage device 116 and/or via the user interface 114. In another embodiment, the OMICS sequencer 112 may be included in or with or integrated into or with the ranking device 102, as illustrated in FIG. 1B.

Upon reception of cell analysis data, the ranking device 102 may score and or rank each cell or cells via the ranking model 110. In an embodiment, the ranking model 110 may be based on a z-score normalization and/or non-parametric or parametric statistical tests that are unpaired or paired, such as Wilcoxon rank sum tests and Wilcoxon signed-rank tests. In such examples, the Wilcoxon rank sum tests and Wilcoxon signed-rank tests may include non-parametric statistical tests that compare data that is not normally distributed (in other includes no variability within one or more groups). The ranking model 110 may utilize a hypothesized mean or median and a biological instance (for example, from the OMICS sequencer 112). The ranking device 102 may produce, generate, or determine a rank or score per each cell or portion of cells. In an embodiment, the rank may be based on cell or gene enrichment or based on the rate or level at which the cells or portions of cells experience the one or more characteristics (for example, the senescence of cells over a selected time). Once scoring and/or ranking is complete, the ranking device 102 may determine a next action. The next action may include one or more of determining a correlation between each analyzed cell and the one or more selected characteristic, suggesting use of a selected product or agent, designing a personalized mixture or product, or prescribing a selected product. Further, the next action and/or the ranking or ranking may be displayed via the user interface 114.

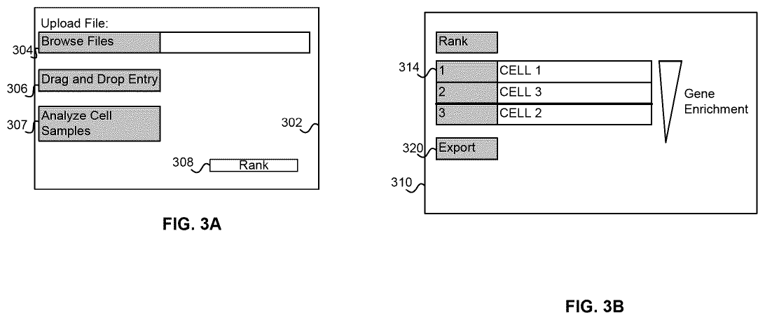

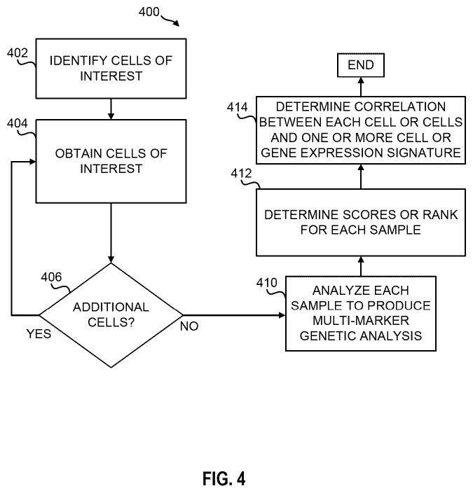

The ranking device 102 of the ranking system 100 (described previously with reference to FIGS. 1A and 1B) may be embodied by one or more computing devices or servers, shown as apparatus 200 in FIG. 2. As illustrated in FIG. 2, the apparatus 200 may include a processor 202, a memory 204, a communications circuitry 206, input-output circuitry 208, analysis circuitry 210, and ranking circuitry 212, each of which will be described in greater detail below. While the various components are only illustrated in FIG. 2 as being connected with processor 202, it will be understood that the apparatus 200 may further comprises a bus (not expressly shown in FIG. 2) for passing information amongst any combination of the various components of the apparatus 200. The apparatus 200 may be configured to execute various operations described above in connection with FIGS. 1A-1B and below in connection with FIGS. 3-4.

The processor 202 (and/or co-processor or any other processor assisting or otherwise associated with the processor) may be in communication with the memory 204 via a bus for passing information amongst components of the apparatus. The processor 202 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 200, remote or “cloud” processors, or any combination thereof.

The processor 202 may be configured to execute software instructions stored in the memory 204 or otherwise accessible to the processor (e.g., software instructions stored on a memory 106, as illustrated in FIGS. 1A-1B). In some cases, the processor may be configured to execute hard-coded functionality. As such, whether configured by hardware or software methods, or by a combination of hardware with software, the processor 202 represent an entity (such as, physically embodied in circuitry) capable of performing operations according to various embodiments of the present disclosure while configured accordingly. Alternatively, as another example, when the processor 202 is embodied as an executor of software instructions, the software instructions may specifically configure the processor 202 to perform the algorithms and/or operations described herein when the software instructions are executed.

Memory 204 is non-transitory and may include, for example, one or more volatile and/or non-volatile memories. In other words, for example, the memory 204 may be an electronic storage device (e.g., a computer readable storage medium). The memory 204 may be configured to store information, data, content, applications, software instructions, or the like, for enabling the apparatus to carry out various functions in accordance with example embodiments contemplated herein.

The communications circuitry 206 may be any means such as a device or circuitry embodied in either hardware or a combination of hardware and software that is configured to receive and/or transmit data from/to a network and/or any other device, circuitry, or module in communication with the apparatus 200. In this regard, the communications circuitry 206 may include, for example, a network interface for enabling communications with a wired or wireless communication network. For example, the communications circuitry 206 may include one or more network interface cards, antennas, buses, switches, routers, modems, and supporting hardware and/or software, or any other device suitable for enabling communications via a network. Furthermore, the communications circuitry 206 may include the processing circuitry for causing transmission of such signals to a network or for handling receipt of signals received from a network.

The apparatus 200 may include input-output circuitry 208 configured to provide output to a user and, in some embodiments, to receive an indication of user input. It will be noted that some embodiments will not include input-output circuitry 208, in which case user input may be received via a separate device such as an OMICS sequencer 112 integrated within the ranking device 102 (shown in FIG. 1B) and/or another separate device. The input-output circuitry 208 may comprise a user interface, such as a display, and may further comprise the components that govern use of the user interface, such as a web browser, mobile application, dedicated client device, or the like. In some embodiments, the input-output circuitry 208 may include a keyboard, a mouse, a touch screen, touch areas, soft keys, a microphone, a speaker, and/or other input/output mechanisms. The input-output circuitry 208 may utilize the processor 202 to control one or more functions of one or more of these user interface elements through software instructions (e.g., application software and/or system software, such as firmware) stored on a memory (e.g., memory 204) accessible to the processor 202.

In addition, the apparatus 200 further comprises an analysis circuitry 210 that, in an embodiment, may initiate analysis of cell samples. The cell samples, as noted, may include cells that exhibit one or more characteristics. The cells may include cells obtained from subjects with one or more selected characteristics (for example, age, race, medical condition, gender, and/or family history). In another embodiment, the analysis circuitry 210 may include equipment or devices that perform the cellular analysis, such as transcriptomics and/or other tests to produce various gene expressions for each cell sample. In yet another embodiment, the analysis circuitry 210 may generate or determine a cell-by gene or cell-by-OMICS matrix based on data received from an analyzer or sequencer, such as an OMICS sequencer and/or other device or storage device. The analysis 210 may utilize processor 202, memory 204, or any other hardware component included in the apparatus 200 to perform these operations, as described in connection with FIGS. 1A-1B above and FIGS. 3-4 below. The analysis circuitry 210 may further utilize communications circuitry 206 to gather data from a variety of sources (such as, a storage device 116 or, as noted, an OMICS sequencer 112) and may utilize input-output circuitry 208 to receive data from a user (such as data related to cell analysis).

In addition, the apparatus 200 further comprises a ranking circuitry 212 that may score each of the analyzed cells and/or rank each of the analyzed cells. The ranking circuitry 212 may utilize one or more statistical models to perform such a scoring and/or ranking. The ranking circuitry 212 may utilize a cell-by-gene or cell-by-OMICS matrix to generate the scores and/or ranking. In such an embodiment, the ranking circuitry 212 may first convert the cell-by-gene matrix into an array. The ranking circuitry 212 may then remove the first column in the array, the first column including, in an example, the agent identities. Next, the ranking circuitry 212 may generate an average of the values in the array and then define the number of instances in the array. The ranking circuitry 212 may then use a statistical model, such as the Wilcoxon sign-rank test, to generate the score and/or rank. The ranking circuitry 212 may utilize processor 202, memory 204, or any other hardware component included in the apparatus 200 to perform these operations, as described in connection with FIGS. 1A-1B above and FIGS. 3-4 below. The ranking circuitry 212 may further utilize communications circuitry 206 to gather data from a variety of sources (such as, from the analysis circuitry 210, storage device 116, and/or, as noted, an OMICS sequencer 112) and may utilize input-output circuitry 208 to receive data from a user (for example, the ranking circuitry 212 may receive data and/or a cell-by gene or cell-by-OMICS matrix from a user interface).

Although components 202-212 are described in part using functional language, it will be understood that the particular implementations necessarily include the use of particular hardware. It should also be understood that certain of these components 202-212 may include similar or common hardware. For example, the analysis circuitry 210 and ranking circuitry 212 may each at times leverage use of the processor 202, memory 204, communications circuitry 206, or input-output circuitry 208, such that duplicate hardware is not required to facilitate operation of these physical elements of the apparatus 200 (although dedicated hardware elements may be used for any of these components in some embodiments, such as those in which enhanced parallelism may be desired). Use of the terms “circuitry,” and “engine” with respect to elements of the apparatus therefore shall be interpreted as necessarily including the particular hardware configured to perform the functions associated with the particular element being described. Of course, while the terms “circuitry” and “engine” should be understood broadly to include hardware, in some embodiments, the terms “circuitry” and “engine” may in addition refer to software instructions that configure the hardware components of the apparatus 200 to perform the various functions described herein.

Although the analysis circuitry 210 and ranking circuitry 212 may leverage processor 202, memory 204, communications circuitry 206, or input-output circuitry 208 as described above, it will be understood that any of these elements of apparatus 200 may include one or more dedicated processor, specially configured field programmable gate array (FPGA), or application specific interface circuit (ASIC) to perform its corresponding functions, and may accordingly leverage processor 202 executing software stored in a memory (such as, memory 204), or memory 204, communications circuitry 206 or input-output circuitry 208 for enabling any functions not performed by special-purpose hardware elements. In all embodiments, however, it will be understood that the analysis circuitry 210 and ranking circuitry 212 are implemented via particular machinery designed for performing the functions described herein in connection with such elements of apparatus 200.

In some embodiments, various components of the apparatus 200 may be hosted remotely (such as, by one or more cloud servers) and thus need not physically reside on the corresponding apparatus 200. Thus, some or all of the functionality described herein may be provided by third party circuitry. For example, a given apparatus 200 may access one or more third party circuitries via any sort of networked connection that facilitates transmission of data and electronic information between the apparatus 200 and the third party circuitries. In turn, that apparatus 200 may be in remote communication with one or more of the other components describe above as comprising the apparatus 200.

As will be appreciated based on this disclosure, example embodiments contemplated herein may be implemented by an apparatus 200. Furthermore, some example embodiments may take the form of a computer program product comprising software instructions stored on at least one non-transitory computer-readable storage medium (for example, memory 204). Any suitable non-transitory computer-readable storage medium may be utilized in such embodiments, some examples of which are non-transitory hard disks, CD-ROMs, flash memory, optical storage devices, and magnetic storage devices. It should be appreciated, with respect to certain devices embodied by apparatus 200 as described in FIG. 2, that loading the software instructions onto a computing device or apparatus produces a special-purpose machine comprising the means for implementing various functions described herein.

Having described specific components of example apparatus 200, example embodiments of the present disclosure are described below in connection with a series of graphical user interfaces and flowcharts.

Turning to FIGS. 3A-3B, a graphical user interface (GUI) is provided that illustrates an interface enabling a user to input data, for example, a cell-by-gene matrix, and generate a ranking based on that data. As noted previously, a user may interact with the ranking system 100 by directly engaging with input-output circuitry 208 of an apparatus 200 comprising a ranking device 102 of the ranking system 100 and/or via the user interface 114. In such an embodiment, the GUIs shown in FIGS. 3A-3B may be displayed to a user by the apparatus 200 or the user interface 114.

Turning to FIG. 3A, a GUI 302 may include one or more inputs enabling a user to input data into the ranking system. For example, the GUI 302 may include a selectable button (see 304) that, when selected, generates a pop-up or separate window with a list of selectable files. In another embodiment, the GUI 302 may include a selectable button (see 306) that, when selected, generates a pop-up or separate window enabling a user to drag a file to the new pop-up or window for upload to the ranking system. In another embodiment, a user may initiate analysis of cell samples based on selection of button 307. In such an embodiment, the cell analysis may be performed once cell samples are received within an analyzer (for example, an OMICS sequencer). The GUI 302 may also include a selectable button (see 308) that when selected causes the ranking system to score and/or rank the received data.

Upon selection of button 308 and subsequent to ranking system scoring and/or ranking the data, a new GUI 310 may be generated. The GUI 310 may include a list of the cells sorted according to rank (see 314). As way of illustration the GUI 310 includes data related to gene enrichment. The cells in 314 may include a identifier corresponding to a selected cell. The GUI 310 may include an export option (see 320). The export option may enable the user to download a copy of the ranking to a file location or desktop and/or to send the results to another user (for example, via e-mail).

In another embodiment, the GUI 310 may further display a percentage of connectivity between the cells and a cell or gene expression signature. The cell or expression signature may correlate to some characteristic of cells and include a corresponding mean expression. For example, a cell signature may include an anti-aging signature. The algorithms described herein may determine whether an overlap exists between the signature and the cell analysis. Any existing overlap may be displayed next to the name of the cell, along with, in some embodiments, an indication of the percentage of overlap.

Turning to FIG. 4, example flowcharts are illustrated that contain example operations implemented by example embodiments described herein. The operations illustrated in FIG. 4 may, for example, be performed by the ranking system 100 and/or the ranking device 102 of the ranking system 100 shown in FIGS. 1A-1B, which may in turn be embodied by an apparatus 200, which is shown and described in connection with FIG. 2. To perform the operations described below, the apparatus 200 may utilize one or more of processor 202, memory 204, communications circuitry 206, input-output circuitry 208, analysis circuitry 210, ranking circuitry 212, and/or any combination thereof. It will be understood that user interaction with the ranking system 100 may occur directly via input-output circuitry 208, or may instead be facilitated by a user interface 114, as shown in FIGS. 1A-1B, and which may have similar or equivalent physical componentry facilitating such user interaction. The order in which the operations are described is not intended to be construed as a limitation, and any number of the described blocks may be combined in any order and/or in parallel to implement the methods.

At block 402, a user or the ranking system 100 may identify cells of interest. The ranking system, for example, may receive a series of cell samples or a selected type of cells. In another embodiment, a user may input a series of cell samples to or into an analyzer. In another embodiment, identification of cells may include determining a type of cells for subsequent testing. For example, skin cells from a suitable subject and a suitable or selected location of the subject may be selected when assessing ranking for a particular characteristic. In a further embodiment, the skin cells may be selected from a selected part of the subject, for example, skin cells from the subject's face, around the subject's eyes, and/or from skin cell's exposed for a prolonged period of time to a selected environment or stimuli. In other embodiments the cells may be exposed to one of a plurality of external stimuli, while some of the cells may be unexposed. At block 404, once cells have been identified, each of the cells may be transferred or provided to an OMICS sequencer or analyzer.

Blocks 402 and 404, as noted, may be an iterative process. For example, a plurality of subjects may provide numerous samples, at varying times, and/or stimuli may be applied to each one of the cell samples in varying concentrations and for selected periods of time. At block 406, if additional cells are available, then the those cells may be obtained and/or external stimuli applied thereto.

At block 410, once all the cell samples have been received or obtained, the cells may be analyzed by an analyzer or sequencer. Such an analysis may provide or generate a multi-marker OMICS analysis, a cell/sample-by-gene or OMICS matrix, and/or other data in one or more formats that include various gene expressions and/or other characteristics. In an embodiment, prior to subsequent steps or processes, the data provided via such analysis may be further processed or converted to a format compatible to scoring or ranking by the ranking system. In other embodiments, the ranking system may convert data to a cell/sample-by-gene or OMICS matrix. In yet another embodiment, the ranking system may receive a cell-by-gene matrix from a user or other data source.

At block 412, the ranking system may determine scores for each cell sample and/or may rank each cell sample based on the applied active agents and/or mixture of active agents. The ranking system may utilize a statistical model and apply such data to the statistical model to generate the scores or ranking. The ranking system may utilize one or more statistical models to produce such scores or ranking.

Finally, at block 414, the ranking system may determine an enrichment and/or depletion between each cell or cells analyzed and one or more cell or gene expression signatures. The correlation may be expressed as a percentage of overlap or similarity. The cell or gene expression signature may be based on a gene expression that exhibits a particular characteristic (for example, a gene expression for a cell that exhibits high anti-aging properties). In an embodiment, rather than or in addition to the determination of the correlation, the method may include determining a next action. The next action may include a determination of a suggested use of a selected product or agent, a personalized mixture or product, and/or prescription a selected product.

In another embodiment, the ranking system may generate a GUI to display the results of the ranking and/or the suggested formula and/or recommended product use. In such embodiments, the GUI may include buttons to export the results and/or the formula or adjustments to a formula. For example, a user may desire to include another sample within the current set of ranked or scored samples. Upon insertion of the sample into a OMICS Sequencer or upon reception of data corresponding to the new sample by the ranking system, the ranking system may re-rank or re-score the samples.

As described above, example embodiments provide methods and apparatuses that enable improved and easily interpretable analysis of cell sample. Example embodiments thus provide tools that overcome the problems faced by a user manually reviewing cell sample analysis, which requires significant time and expertise, while reducing potential human error (for example, miss-ranking or miss-scoring cells in relation to characteristics). Moreover, embodiments described herein avoid misinterpretation and eliminate the need for expertise in understanding selected cell analysis charts (such as a heat map). As these examples all illustrate, example embodiments contemplated herein provide technical solutions that solve real-world problems faced during interpretation of cell analysis.

FIG. 4 illustrates operations performed by apparatuses, methods, and computer program products according to various example embodiments. It will be understood that each flowchart block, and each combination of flowchart blocks, may be implemented by various means, embodied as hardware, firmware, circuitry, and/or other devices associated with execution of software including one or more software instructions. For example, one or more of the operations described above may be embodied by software instructions. In this regard, the software instructions which embody the procedures described above may be stored by a memory of an apparatus employing an embodiment of the present disclosure and executed by a processor of that apparatus. As will be appreciated, any such software instructions may be loaded onto a computing device or other programmable apparatus (e.g., hardware) to produce a machine, such that the resulting computing device or other programmable apparatus implements the functions specified in the flowchart blocks. These software instructions may also be stored in a computer-readable memory that may direct a computing device or other programmable apparatus to function in a particular manner, such that the software instructions stored in the computer-readable memory produce an article of manufacture, the execution of which implements the functions specified in the flowchart blocks. The software instructions may also be loaded onto a computing device or other programmable apparatus to cause a series of operations to be performed on the computing device or other programmable apparatus to produce a computer-implemented process such that the software instructions executed on the computing device or other programmable apparatus provide operations for implementing the functions specified in the flowchart blocks.

The flowchart blocks support combinations of means for performing the specified functions and combinations of operations for performing the specified functions. It will be understood that individual flowchart blocks, and/or combinations of flowchart blocks, can be implemented by special purpose hardware-based computing devices which perform the specified functions, or combinations of special purpose hardware and software instructions.

In some embodiments, some of the operations above may be modified or further amplified. Furthermore, in some embodiments, additional optional operations may be included. Modifications, amplifications, or additions to the operations above may be performed in any order and in any combination.

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A method for ranking a cells of interest, the method comprising:
identifying cells of interest of a subject;
analyzing, via an OMICS sequencer, each portion of the cells to produce a multi-marker OMICS analysis;
determining a rank for each of the cells for one or more selected characteristics based on application of the multi-marker OMICS analysis to an expression model for the one or more selected characteristics; and
determining a next action based on a the rank for each of the cells.

2. The method of claim 1, wherein determining the rank comprises:
converting the multi-marker genetic or OMICS analysis to an array of values;
removing identifiers from the array of values;
determining an average of the array of values and a number of instances in the array of values;
applying the array of values to assigned pair ranked test to generate a score for each value in the array of values based on a signature of the one or more selected characteristics; and
re-assigning the identifiers to the array of values.

3. The method of claim 1, wherein the OMICS sequencer comprises one of a genomics sequencer, transcriptomics sequencer, proteomics sequencer, epigenomics sequencer, lipidomics sequencer, or metabolomics sequencer; wherein the multi-marker OMICS analysis comprises one of transcriptomics data, proteomics data, epigenomics data, genomics data, lipidomics data, or metabolomics data; wherein the cells of interest comprise one or more of skin cells, skin tissue, hair cells, scalp cells, scalp tissue, reconstructed tissue, or ex vivo skin; and wherein the one or more cells of interest are collected from one or more subjects of one or more different ages, one or more difference races, one or more genders.

4. The method of claim 1, further comprising:
in response to determination of the rank for each of the cells:
generating a user interface;
displaying each of the cells based on ranking; and
displaying data indicative of the correlation between each analyzed cell and the one or more selected characteristics.

5. The method of claim 1, wherein the next action comprises one or more of determining a correlation between each analyzed cell and the one or more selected characteristic, suggesting use of a selected product or agent, designing a personalized mixture or product, or prescribing a selected product.

6. The method of claim 1, wherein the one or more selected characteristics comprise one or more of differentiation, keratinization, immune response, angiogenesis, melanogenesis, autophagy/mitophagy, senescence, longevity, hair growth and health, microbiome, DNA repair, epigenetics, proteostasis, intercellular communication, scalp health, nutrient signaling, inflammation, wound healing response, oxidative stress response, proliferation, stem cell renewal, clonogenicity, or skin barrier health.

7. The method of claim 1, further comprising:
prior to analysis of each portion of the cells, iteratively applying different stimuli to different portion of the cells; wherein the stimuli induces a pathological state for each different portion of cells; and wherein the stimuli comprises one or more of exposure to sunlight, exposure to ultraviolet light, exposure to environmental pollutants, exposure to chemicals, exposure to antibodies, exposure to vesicles, exposure to genetic stimuli, exposure to a selected temperature, exposure to a selected pressure, or a selected time.

8. An apparatus for ranking a cells of interest, the apparatus comprising:
an analysis circuitry configured to:
obtain a multi-marker OMICS analysis of a plurality of cells, each of the plurality of cells exhibiting one or more characteristics; and
a ranking circuitry configured to:
determine a rank for each of the cells for the one or more selected characteristics based on application of the multi-marker OMICS analysis to an expression model for the one or more selected characteristics, and
determine cell enrichment for each analyzed cells based on the rank for each of the cells.

9. The apparatus of claim 8, further comprising an OMICS sequencer configured to analyze cells to produce the multi-marker OMICS analysis.

10. The apparatus of claim 8, wherein the analysis circuitry communicatively connects to the OMICS sequencer and wherein the analysis circuitry is further configured to, in response to reception of cells of interest at the OMICS sequencer, prompt the OMICS sequencer to analyze the cells of interest.

11. The apparatus of claim 8, further comprising a user interface communicatively connected to the analysis circuitry and wherein the analysis circuitry obtains the multi-marker OMICS analysis from the user interface.

12. The apparatus of claim 8, wherein the cells of interest comprise cells obtained from a subject, and wherein the ranking circuitry is further configured to determine a recommended use suggestion of a selected product based on history of use by the subject.

13. A computer program product for ranking a cells of interest, the computer program product comprising a non-transitory machine-readable storage medium storing software instructions that, when executed, cause an apparatus to:
obtain a multi-marker OMICS analysis of a plurality of cells, each of the plurality of cells comprising one or more of cells subjected to an external stimuli or cells exhibiting one or more of a plurality of selected cell characteristics; and
determine a rank for each of the cells for one or more selected characteristics based on application of the multi-marker OMICS analysis to an expression model for the one or more selected characteristics.

14. The computer program product of claim 12, wherein the non-transitory machine-readable storage medium stores additional software instructions that, when executed, determine a recommended use suggestion of a selected product based on history of use on the cells.

15. The computer program product of claim 14, wherein the multi-marker OMICS analysis comprises one or more of a heatmap, a cell or sample by OMICS matrix, or a cell type distribution.
